(19) United States
(12) Patent Application Publication
(10) Pub. No.: US 2013/0157885 A1
(43) Pub. Date: Jun. 20, 2013

(54) SYSTEM AND METHODS FOR SELECTIVE MOLECULAR ANALYSIS

(71) Applicant: RHEONIX, INC., Ithaca, NY (US)

(72) Inventor: Gwendolyn Spizz, Ithaca, NY (US)

(73) Assignee: RHEONIX, INC., Ithaca, NY (US)

(21) Appl. No.: 13/680,231

(22) Filed: Nov. 19, 2012

Related U.S. Application Data
(60) Provisional application No. 61/561,063, filed on Nov. 17, 2011.

(51) Int. Cl. C12Q 1/68

(52) U.S. Cl. CPC C12Q 1/6874
USPC 506/9; 435/6.11; 435/287.2

(57) ABSTRACT

Methods and systems for selectively amplifying a target DNA sequence in the presence of non-target DNA sequence in a sample, comprising: contacting the sample with an oligonucleotide system under hybridization conditions to form a reaction mixture including a forward primer and a reverse primer, wherein either the forward or reverse primer is modified to preferentially increase hybridization between the primer and the target sequence; cycling the hybridization of the oligonucleotide system so that, if the target DNA sequence is present in the sample, the primers hybridize to the target DNA sequence and the reaction mixture results in a first amplified product; and detecting the first amplified product.

### CROSS-REFERENCE TO RELATED APPLICATIONS

This application claims priority to U.S. Provisional Patent Application Ser. No. 61/561,063, filed on Nov. 17, 2011 and entitled “Selective Molecular Analysis System,” the entire disclosure of which is incorporated herein by reference.

### BACKGROUND

1. Field

This specification relates to the field of molecular biology and, more specifically, to methods and systems for selectively analyzing particular alleles of a gene or genes, and applications thereof.

2. Description of the Related Art

Genes are determined to be either “wild-type” or “mutant” based upon variations in a particular gene's sequence of nucleotide bases. The wild-type gene is generally the first identified sequence of a particular gene or the most commonly occurring sequence of a particular gene. A mutant is any gene that has a sequence that varies by at least one nucleic acid base from the sequence of the wild-type gene. There may be one or many mutations for any particular gene and there often is more than one mutation for any particular gene. In certain cases it is important to identify particular mutations for particular genes. The mutations in these cases are generally inherited through and occur in all the somatic cells of an organism.

Germline mutations are inherited and are present in all cells within the organism, and as a result homozygous wild-type or mutant sequences will be present in 100% of the total DNA of that organism, and heterozygous sequences will generally be present in 50% of the total DNA of that organism. In contrast, somatic mutations occur in individual cells in the organism and may occur at any time during the lifetime of that particular organism. As a result, there will be a heterozygous genotype within the cell and its progeny. Often, DNA containing the mutation will represent extremely small percentages of the total DNA from the entire specimen, and may be as low as or less than 1%. This very small incidence of the mutation may not be selectively noticeable using classical molecular biological techniques used for allelic analysis.

Therefore systems and method that can selectively identify only particular alleles in a mixture of nucleic acids purified from a sample of cells from a particular single organism and then analyze those samples for particular mutations of interest would be very useful for researchers and clinical professionals.

### BRIEF SUMMARY

In accordance with the foregoing objects and advantages are described methods and systems for selectively analyzing particular alleles of a gene or genes, and applications thereof.

According to one aspect, a method for selectively amplifying a target DNA sequence in the presence of non-target DNA sequence in a sample, the method comprising: (i) contacting an oligonucleotide system with the sample under hybridization conditions to form a reaction mixture, the oligonucleotide system including a forward primer and a reverse primer, wherein one of the forward or reverse primer is modified to preferentially increase hybridization between the primer and the target sequence, the modification comprising a modified 3′ terminal nucleotide; (ii) cycling the oligonucleotide system so that, if the target DNA sequence is present in the sample, the forward primer and the reverse primer hybridize to the target DNA sequence and the reaction mixture results in a first amplified product; and (ii) detecting the first amplified product, wherein the detecting step comprises use of a target DNA probe component for detecting the target DNA sequence, the target DNA probe component comprising a first modification, wherein the first modification preferentially increases hybridization between the target DNA probe component and the first amplified product.

According to a second aspect is the above method, wherein the modified 3′ terminal nucleotide is selected from the group consisting of locked nucleic acid, cLNA, bridged nucleic acid, zip nucleic acid, minor groove binder, peptide nucleic acid, and combinations thereof.

According to a second aspect is the above method, the modified primer further comprises one or more additional modified nucleotides.

According to a third aspect is the above method, wherein the modified primer comprises the oligonucleotide sequence 5′-XYZ-3′, wherein: (i) X comprises one or more biotin groups; (ii) Y comprises one or more nucleic acid bases; and (iii) Z comprises one or more modified nucleotides. According to an aspect, Z comprises at least one modified nucleotide selected from the group consisting of locked nucleic acid, cLNA, bridged nucleic acid, zip nucleic acid, minor groove binder, peptide nucleic acid, and combinations thereof. According to another aspect, Z comprises two consecutive locked nucleic acids at the 3′-terminus of the modified primer.

According to a fourth aspect is the above method, wherein the modified primer comprises the oligonucleotide sequence 5′-YZYZ-3′, wherein: (i) Y comprises one or more nucleotides; and (ii) Z comprises one or more modified nucleotides.

According to a fifth aspect is the above method, wherein the target DNA sequence and at least some of the non-target DNA sequence in the sample differ by only one nucleic acid.

According to a sixth aspect is the above method, wherein the step of detecting the first amplified product comprises the steps of: (i) providing a microarray comprising a set of features including the target DNA probe component for detecting the target DNA sequence, and further including a component intended to serve as a positive control and a component intended to serve as a negative control; (ii) contacting the microarray with the cycled reaction mixture to enable the first amplified product to bind to the target DNA probe component, wherein such binding results in the feature emitting the detectable signal; and (iii) detecting the emitted detectable signal.

According to another aspect, a method for selectively amplifying a target DNA sequence in the presence of non-target DNA sequence in a sample, wherein the target DNA sequence and at least some of the non-target DNA sequence in the sample differ by only one nucleic acid, the method comprising: (i) contacting an oligonucleotide system with the sample under hybridization conditions to form a reaction mixture, the oligonucleotide system including a forward primer and a reverse primer, wherein one of the forward or reverse primer is modified to preferentially increase hybridization between the primer and the target sequence, the modification comprising: (i) a modified 3′ terminal nucleotide, and (ii) one or more additional modified nucleotides; (ii) cycling the oligonucleotide system so that, if the target DNA sequence is present in the sample, the forward primer and the reverse primer hybridize to the target DNA sequence and the reaction mixture results in a first amplified product; (iii) providing a microarray comprising a set of features including at least a target DNA probe component for detecting the target DNA sequence, and further including a component intended to serve as a positive control and a component intended to serve as a negative control, wherein the target DNA probe component comprises a first modification, wherein the first modification preferentially increase hybridization between the target DNA probe component and the first amplified product; (iv) contacting the microarray with the cycled reaction mixture to enable the first amplified product to bind to the target DNA probe component, wherein such binding results in the feature emitting the detectable signal; and (v) detecting the emitted detectable signal; (vi) wherein the target DNA probe component comprises a first modification, wherein the first modification preferentially increases hybridization between the target DNA probe component and the first amplified product.

According to another aspect, a system for selectively amplifying a target DNA sequence, the system comprising: (i) a sample comprising a non-target DNA sequence and potentially comprising the target DNA sequence; (ii) an oligonucleotide system comprising a forward primer and a reverse primer under hybridization conditions to form a reaction mixture, wherein one of the forward or reverse primer is modified to preferentially increase hybridization between the primer and the target sequence, the modification comprising a modified 3′ terminal nucleotide; (ii) a thermocycler adapted to cycle the oligonucleotide system so that, if the target DNA sequence is present in the sample, the forward primer and the reverse primer hybridize to the target DNA sequence and the reaction mixture results in a first amplified product; (iii) a target DNA probe component for detecting the target DNA sequence, the target DNA probe component comprising a first modification, wherein the first modification preferentially increases hybridization between the target DNA probe component and the first amplified product; and (iv) a detector adapted to detect the first amplified product.

According to another aspect, modified 3′ terminal nucleotide is selected from the group consisting of locked nucleic acid, cLNA, bridged nucleic acid, zip nucleic acid, minor groove binder, peptide nucleic acid, and combinations thereof.

According to another aspect, the modified primer further comprises one or more additional modified nucleotides.

According to another aspect, the modified primer comprises the oligonucleotide sequence 5′-XYZ-3′, wherein: X comprises one or more biotin groups; Y comprises one or more nucleotides; and Z comprises one or more modified nucleotides. According to one aspect, Z comprises two consecutive locked nucleic acids at the 3′-terminus.

According to another aspect, the target DNA sequence and at least some of the non-target DNA sequence in said sample differ by only one nucleic acid.

According to another aspect, the detector is a microarray.

According to another aspect, the microarray comprises a target DNA probe component for detecting the target DNA sequence, a component intended to serve as a positive control, and a component intended to serve as a negative control.

According to another aspect, the modified primer comprises the oligonucleotide sequence 5′-YZYZ-3′, wherein: Y comprises one or more nucleotides; and Z comprises one or more modified nucleotides.

According to another aspect, the target DNA probe component comprises at least one modified nucleotide selected from the group consisting of locked nucleic acid, cLNA, bridged nucleic acid, zip nucleic acid, minor groove binder, peptide nucleic acid, and combinations thereof.

### BRIEF DESCRIPTION OF THE SEVERAL VIEWS OF THE DRAWING(S)

The present specification will be more fully understood and appreciated by reading the following Detailed Description in conjunction with the accompanying drawings, in which:

FIG. 1A is a schematic of a PCR reaction for detection of a wild-type or mutant allele (SEQ ID NO:1) according to existing methods;

FIG. 1B is an expanded view of the boxed region (SEQ ID NO:2) in FIG. 1A;

FIG. 2 is schematic of an assay for detection of wild-type and/or mutant alleles of the KRAS gene according to existing methods (including a targeted KRAS region (SEQ ID NO:3), and the translation for a portion of the protein (SEQ ID NO: 4));

FIG. 3 shows the results of microarray analysis using a microarray as organized in the table, where dots represent hybridization;

FIG. 4A is schematic of an assay for detection of wild-type and/or mutant alleles of the KRAS gene according to an embodiment (including a targeted KRAS region (SEQ ID NO:5), and the translation for a portion of the protein (SEQ ID NO: 6));

FIG. 4B is an expanded view of the boxed region in FIG. 4A (including a targeted KRAS region (SEQ ID NO:7), and the translation for a portion of the protein (SEQ ID NO: 8));

FIGS. 5A and 5B are tables depicting the sequences of the primers shown in FIGS. 4A and 4B according to an embodiment;

FIG. 6A shows the results of a microarray analysis using a microarray as organized in FIG. 6B and using the amplicons from the conditions described in part in FIGS. 4A through 5B, where dots represent hybridization;

FIG. 6B is a table depicting the organization of the microarray in FIG. 6A;

FIGS. 7A and 7B are tables depicting the sequences of the primers used for a set of experiments according to an embodiment;

FIG. 8A shows the results of a microarray analysis using a microarray as organized in FIG. 8B and using the amplicons from the conditions described in part in FIGS. 7A and 7B, where dots represent hybridization;

FIG. 8B is a table depicting the organization of the microarray in FIG. 8A;

FIGS. 9A and 9B are schematics for PCR amplification for detection of a wild-type or mutant BRAF allele according to an embodiment (SEQ ID NO:24);

FIG. 10A is a table depicting the sequences of the primers used for the KRAS 38G>A assay (results shown in FIGS. 11A and 11B) and 10B is a table depicting the sequences of the primers used for the BRAF1799T>A assay (results shown in FIGS. 11A and 11B, according to an embodiment;

FIG. 11A shows the results of a microarray analysis using a microarray as organized in FIG. 11B and using the amplicons from the conditions described in part in FIGS. 9A through 10B, where dots represent hybridization; and

FIG. 11B is a table depicting the organization of the microarray in FIG. 11A.

### DETAILED DESCRIPTION

According to one aspect of the invention is a method and system to selectively amplify only a specific allele of a particular gene. For example, described herein are methods and systems that use, in part, a ‘nucleic acid substitute’ to selectively amplify and/or to selectively detect specific alleles such as mutant alleles.

According to one embodiment, the ‘nucleic acid substitute’ can be any of a wide variety of substitutes, including but not limited to Locked Nucleic Acids (“LNA”), cLNA's, Bridged Nucleic Acids (“BNA”), Zip Nucleic Acids (“ZNA”), Minor Groove Binders (“MGB”), Peptide Nucleic Acids (“PNA”), incorporated into amplification primers or Allele Specific PCR (“asPCR”), Mutant Allele Specific Amplification (“MASA”), and/or DNA Duplex stabilizing chemistry, among others.

According to one embodiment of the system or method, the primers used to amplify the genetic sequence are designed such that they only anneal to particular genetic sequences of specific alleles, thus only amplifying those particular alleles. As just one example of the system or method, one would design a primer (or multiple primers, or a primer for each mutation of interest) that does not amplify the wild-type gene (or that selects against any particular known allele of a gene), but that does amplify a particular allele of the same gene. The resulting amplicons generated from the selective amplification may then be applied to a microarray that incorporates probes for particular alleles of the gene. In one embodiment, the probes also use a ‘nucleic acid substitute’ such as LNA's, cLNA's, BNA's, ZNA's, MGB's, and/or PNA's to increase fidelity and improve hybridizing specificity, including probes for the wild-type allele or any allele selected against by primer design. The microarray is then analyzed for which probes hybridized with which amplicons. In the case where an amplicon hybridizes with a wild-type probe or the probe for the allele selected against, the primer either failed or was not properly constructed to select against the amplification of the wild-type allele or any other allele selected against. In a preferred embodiment, little to no amplicon would hybridize with the wild-type probe (or the probe for the allele selected against), thus indicating successful selectivity of the primer. In the case where there is very little or no amplicon hybrized with the wild-type probe (or any other allele selected against), the remaining probes with sequences that are complimentary to particular mutations of the wild-type gene are analyzed. If there are positive hybridizations then the initial cells in the sample included some cells in which genetic or somatic mutations occurred. Information about a particular mutation(s) within a population of cells can be useful for, for example, research concerning particular diseases and their progression. In the clinical setting, the information may inform a clinician about a course of treatment for a particular disease of disease state.

According to another embodiment, the method or system is useful in the case where only a small amount of genetic material in a particular sample contains a mutation. For example, when a tumor is biopsied the resulting tissue may only contain a small percentage of cells that contain a particular mutation in their genetic makeup while the vast remainder of the population is still wild-type or the original germ line sequence. The methods and systems described herein can be used to selectively prevent amplification of the background wild-type or original germ line sequence allele and thereby its later analysis, thereby increasing the signal from the mutations that may be present in the original sample. Identifying mutations at an early stage, for example when they represent only a small fraction of the cells in a tumor, can provide researchers with valuable information concerning particular diseases and their progression and clinicians with more and often more effective options for the treatment of a disease.

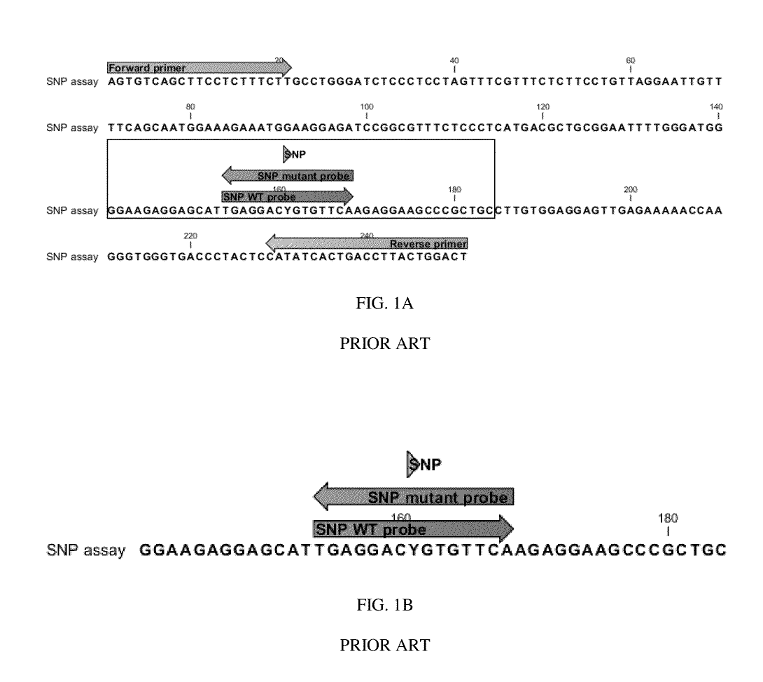

Referring now to the drawings, wherein like reference numerals refer to like parts throughout, there is seen in FIGS. 1A and 1B a method and system for traditional PCR amplification and probe analysis. The amplified genetic sequences may be wild-type, may vary in some way (such as single nucleotide polymorphisms or SNPs), or may be mutated in some way. Unfortunately, since mutations are often simply just a single base pair difference from the wild-type allele, the ability to differentiate sequences is very difficult since the two oligonucleotide strands will typically have nearly identical physical properties. That is, a complex mixture containing both mutated and wild-type (or non-mutated) template result in very similar amplicons following PCR amplification; these similar amplicons will behave very similarly because they may differ by no more than a single base. One property that these similar amplicons will have in common is that they may both hybridize with a target probe sequence regardless of their slight sequence difference, and therefore an analyst may not be able to distinguish between the two different oligonucleotides. The physical characteristics most commonly similar between alleles are the melting temperature (“Tₘ”) of the hybridized pair or the molecular weight of the allele, among other properties known in the art. A further complication occurs when the mutation is not inherited through an organism's germ line and therefore exists in every cell in the organism, but when the mutation occurs spontaneously in a cell in the body of the organism and therefore may exist in only certain cells (often less than 1%) of the cells of a sample from the organism. In the case of a somatic mutation, classical SNP analysis does not provide useful information since the overwhelming amount of DNA in the sample does not contain the somatic mutation of interest. In this situation, as described above, the classical system or method of analysis will not adequately amplify the mutation since it will generally amplify the majority allele.

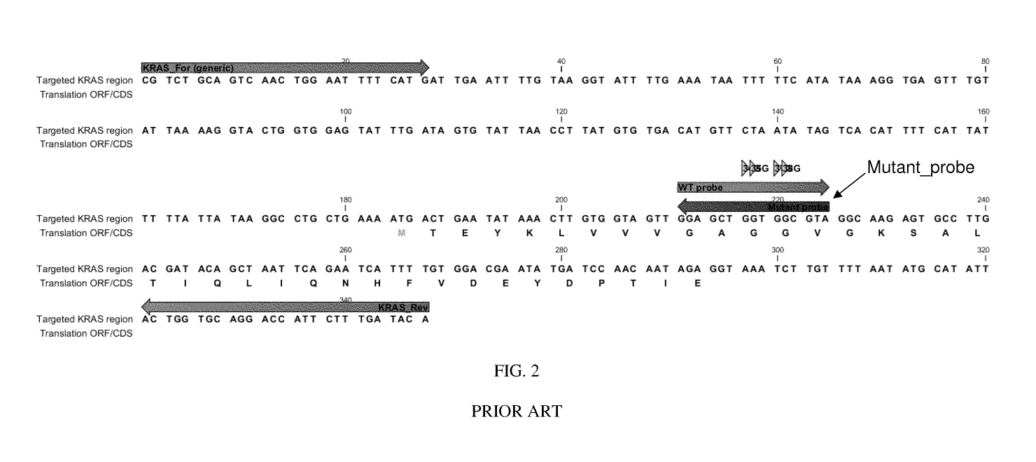

FIG. 2 is a schematic of a KRAS assay, in which the prior art system or method is used to amplify and probe the KRAS gene, which is either wild-type or a variant allele. All or a portion of the KRAS gene locus is amplified using traditional probes including the primer pair “KRAS_For” and “Mutant probe,” and the primer pair “WT_probe” and “KRAS_Rev.”

KRAS, also known as “GTPase KRas” or “V-Ki-ras2 Kirsten rat sarcoma viral oncogene homolog,” is a protein in humans encoded by the KRAS gene. KRAS is a proto-oncogene (a normal gene that can become an oncogene due to mutation(s), increased expression, or other activation) in which a single amino acid substitution—resulting from a single nucleotide substitution—is responsible for activating the oncogene activity of KRAS. KRAS has been implicated in various types of cancer, including but not limited to lung adenocarcinoma, mucinous adenoma, ductal carcinoma of the pancreas, and colorectal carcinoma, among others. Indeed, mutations in the KRAS gene are estimated to occur in over 90% of pancreatic cancers. Mutations are typically found to affect codons 12, 13, and 61 of KRAS protein, which prevent GTP-GDP exchange, keeping KRAS in the constitutively active GTP bound state. Further, activating mutations in the KRAS gene are associated with poor response to anti-epidermal growth factor receptor (“EGFR”) response. Accordingly, testing for these activation mutations can be an important aspect of anti-EGFR therapy. The presence of a KRAS mutation in a population of cells (such as a tumor) is currently performed through various methods, including real-time PCR and monoclonal antibody tests.

Other oncogenes and proto-oncogenes that could be analyzed include, but are not limited to, NFKB2, NRAS, BCL2, BCL3, BCL6, BRAF, PIM1, IRF4, JUN, LCK, RAF1, MAFB, DDB2, DEK, SMO, ROS1, TET2, NTRK1, FGFR2, EGFR, ERBB2, and MYB, among many others. Further, many other types of genes, alleles, or other locations throughout an organism's DNA complement can be probed and analyzed using the systems and methods described herein.

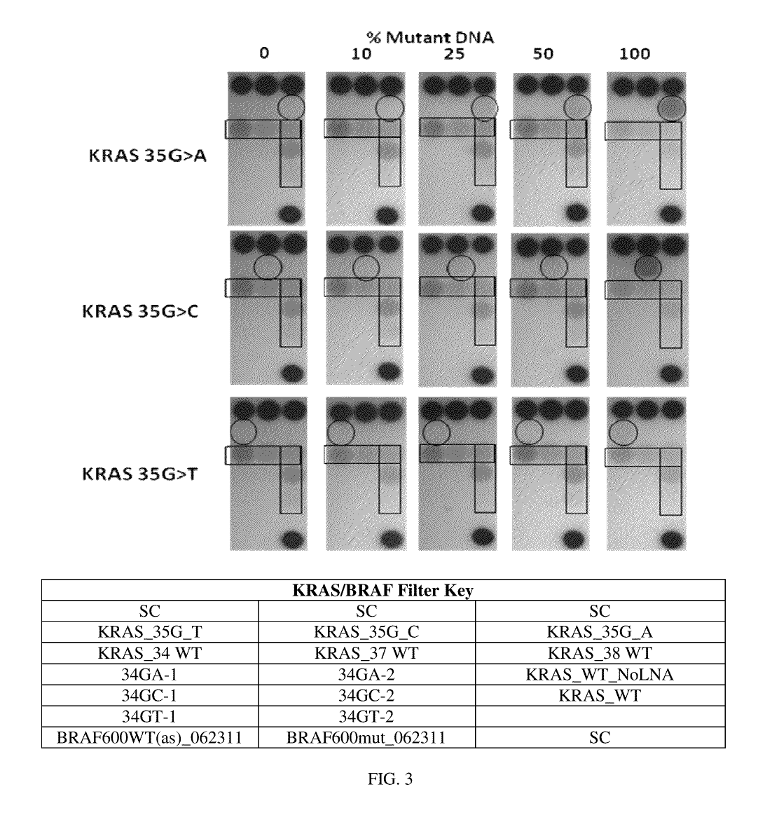

The resulting amplicons generated from the traditional amplification method depicted in FIG. 2 are then applied to a microarray that incorporates probes for particular alleles of the gene, as shown in FIG. 3. The microarray is then analyzed for which probes hybridized with which amplicons. For the experiment depicted in FIGS. 2 and 3, the amount of “Mutant DNA” is a percentage of the template, ranging from 0% to 100% of mutant allele (either the KRAS G35A allele, KRAS G35C allele, or KRAS G35T allele), with the remaining percentage (100%, 90%, 75%, 50%, and 0%) being the wild-type KRAS allele. The resulting amplicons are then run against a microarray with specific probes. For example, the mutant allele amplicons are to be detected with one or more mutant probes (e.g., KRAS_{—}35G_T, KRAS_{—}35G_C, or KRAS_{—}35G_A), and the wild-type allele amplicons are to be detected with each of the wild-type probes.

The probes, which can be part of a microarray or one of several other detection mechanisms, can be unmodified or natural oligos, or, alternatively, can comprise one or more modifications. According to a preferred embodiment, a probe is designed with one or more modifications, including but not limited to any of the modifications described herein, in order to increase the Tm difference and thus increase selectivity. The modification(s) may be anywhere along the probe.

FIG. 3 depicts the results of a series of experiments using traditional amplification methods to detect the presence of mutant alleles in a mutant/wild-type DNA mixture ranging from 0%/100% to 100%/0%. At less than 100% of mutant DNA, each of the three different mutant alleles is nearly impossible to detect. For example, the 0%, 10%, 25%, and 50% Mutant DNA columns reveal almost no detectable mutant amplicon. And for KRAS 35G>T, there is no detection even at 100% Mutant DNA. This detection method would not be feasible for most tumor analysis, since the percentage of cells with a mutated version of the KRAS gene will almost certainly be at a significantly lower percentage.

### Example 1

### KRAS G35X Analysis Using Modified Primers and Probes

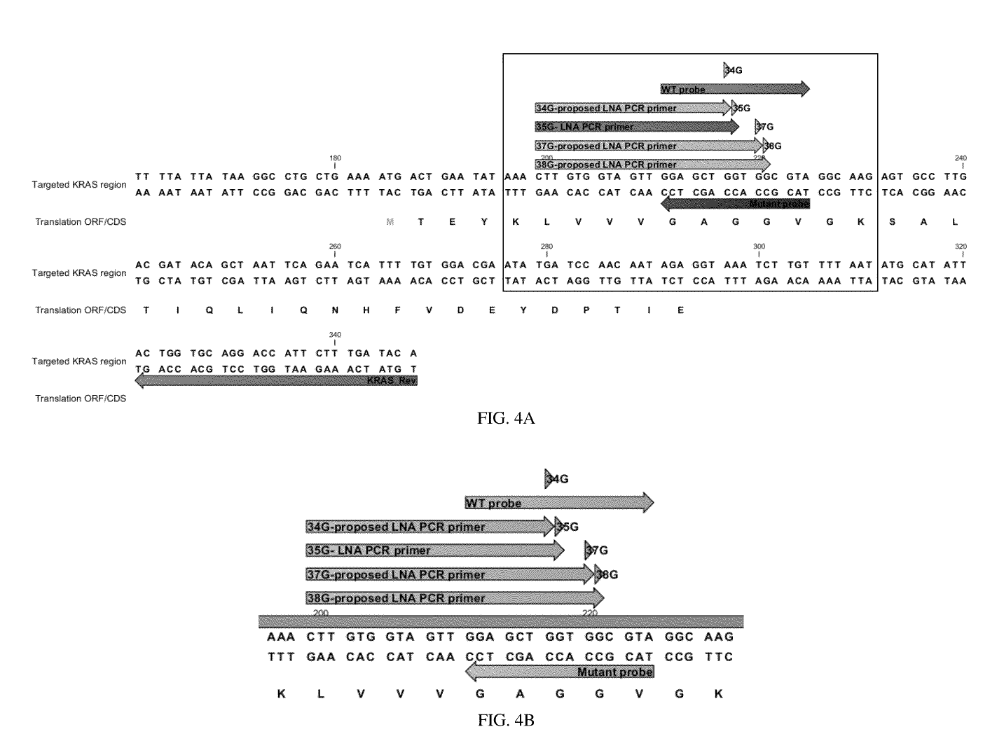

FIGS. 4A through 6B depict the conditions and results of a KRAS assay using modified primers and modified probes according to an embodiment. In this assay, KRAS is amplified using a mixture of biotinylated KRAS_LNA PCR primers specific to the site of interest (for example, the “34G-proposed LNA PCR primer,” “35G-LNA PCR primer,” “37G-proposed LNA PCR primer,” and the “38G-proposed LNA PCR primer,” all depicted in FIGS. 4A and 4B) and biotinylated KRAS Rev primers (for example, “KRAS_Rev”), as shown in FIGS. 4A and 4B. Capture probes with a wild-type “G” at the individual targeted positions are made using the coding strand sequence and will hybridize with the amplified biotinylated non-coding strand. Capture probes that will recognize mutant A, C, or T at the targeted positions are made using the non-coding strand sequence (therefore, T, G, or A at the respective sites) and will hybridize with the amplified biotinylated coding strand. According to one embodiment, the biotinylated LNA modified mutant primers and LNA modified capture probes are made to opposite strands. According to one embodiment, the biotinylated wild-type (generic primer) can be made off of the same strand as the mutant capture probes, and wild-type capture probe made off of same strand as the mutant primers.

Although this and other examples herein use biotinylated primers, the primers may be absent biotinylation, or may be otherwise modified. For example, one of skill in the art would recognize that there are numerous other types of modification, including but not limited to fluorescence, isotopic labeling, antibodies, and quantum dots, among many others.

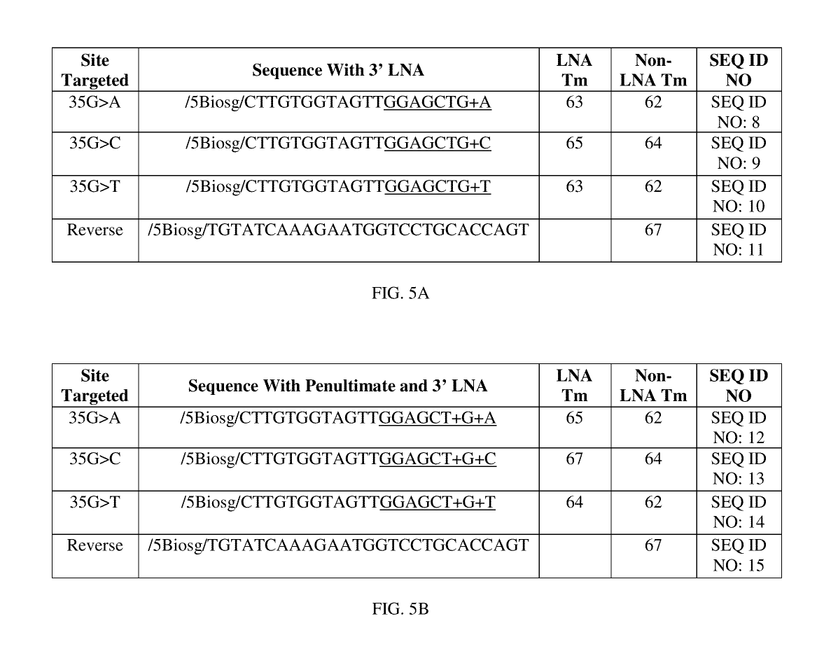

As depicted in FIG. 5A, the following primers were used for amplification of a partial sequence within KRAS gene (the KRAS assay):

Forward Primers:
(SEQ ID NO: 8)
/5Biosg/CTTGTGGTAGTTGGAGCTG+A
for 35G > A detection;
(SEQ ID NO: 9)
/5Biosg/CTTGTGGTAGTTGGAGCTG+C
for 35G > C detection;
(SEQ ID NO: 10)
/5Biosg/CTTGTGGTAGTTGGAGCTG+T
for 35G > T detection;
Reverse Primer:
(SEQ ID NO: 11)
/5Biosg/TGTATCAAAGAATGGTCCTGCACCAGT
for all reactions.

where “/5Biosg/” represents a biotin, the “+[A, C, or T]” represents an LNA, and underlining indicates homology with the microarray probe. The Tm depicted in FIGS. 5A and 5B were calculated using traditional methods. According to a preferred embodiment, the modification of the 3′ terminal base with an LNA provides selective amplification of the mutation at site 35 while not amplifying the wild-type form of the allele.

In another embodiment, as depicted in FIG. 5B, the following primers were used for amplification of a partial sequence within KRAS gene (the KRAS assay):

Forward Primers:
(SEQ ID NO: 12)
/5Biosg/CTTGTGGTAGTTGGAGCT+G+A
for 35G > A detection;
(SEQ ID NO: 13)
/5Biosg/CTTGTGGTAGTTGGAGCT+G+C
for 35G > C detection;
(SEQ ID NO: 14)
/5Biosg/CTTGTGGTAGTTGGAGCT+G+T
for 35G > T detection;
Reverse Primer:
(SEQ ID NO: 15)
/5Biosg/TGTATCAAAGAATGGTCCTGCACCAGT
for all reactions.

where “/5Biosg/” represents a biotin, the “+[A, C, or T]” represents an LNA, and underlining indicates homology with the microarray probe. These primers/probes differ from those depicted in FIG. 5A in that there are two terminal LNAs rather than just one. As shown in the “LNA Tm” columns in FIGS. 5A and 5B, this additional LNA further affects the Tm of the primers.

Although this example depicts two terminal LNAs, the “+[A, C, or T]” could be any other modification described herein or known to one of skill in the art. Further, the one or more modification(s) in addition to the modified nucleotide at the 3′ terminal end could be anywhere along the primer. For example, a primer sequence for 35G>A detection could be any of the following:

(SEQ ID NO: 30)
CTTGTGGTAGTTGGAGC+TG+A for 35G > A detection;
(SEQ ID NO: 31)
CTTGTGGTAGTTGGAG+CTG+A for 35G > A detection;
(SEQ ID NO: 32)
CTTGTGGTAGTTGGA+GCTG+A for 35G > A detection;
(SEQ ID NO: 33)
CTTGTGGTAGTTGG+AGCTG+A for 35G > A detection;
(SEQ ID NO: 34)
CTTGTGGTAGTTG+GAGCTG+A for 35G > A detection;

Further, there can be multiple modifications in order to further maximize selectivity. For example, a primer sequence for 35G>A detection could be any of the following, among many other variations:

(SEQ ID NO: 35)
CTTGTGGTAGTTGGAGC+T+G+A
for 35G > A detection;
(SEQ ID NO: 36)
CTTGTGGTAGTTG+G+A+G+C+T+G+A
for 35G > A detection;

The design of the primer will depend at least in part on the requirements of the system—including but not limited to the target DNA sequence—necessary to sufficiently alter the Tm difference and thereby increase selectivity.

As described above, the primers used for amplification of the KRAS allele in this example are modified by biotin and one or more Locked Nucleic Acids (“LNA”). An LNA is a modified nucleotide with an extra bridge connecting the 2′ oxygen and 4′ carbon. That bridge effectively “locks” the ribose in the 3′-endo conformation, thereby increasing the melting temperature of an oligonucleotide (compare, for example, the “LNA Tm” and “Non-LNA Tm” columns in FIGS. 5A and 5B).

Although the primers used for amplification of the KRAS allele in this example are modified by biotin and one or more LNA, the primers and/or probes can be modified in a variety of other ways in order to increase hybridization. This includes, but is not limited to, the incorporation into the primer and/or probe of one or multiple LNAs, cLNA's, BNAs, ZNAs, MGBs, and/or PNAs, among many others. The beneficial result is that when a sample of cells that contain a somatic mutation are analyzed, only the allele(s) of interest is amplified and therefore subsequent analysis is facilitated since the material being analyzed only contains the allele(s) of interest. In conjunction with modifying primers to selectively amplify particular known alleles of a gene a probe system (such as a microarray, fluorescence, enzymatic systems) may also contain modified probe sequences (using Locked Nucleic Acids (LNA's), cLNA's, Bridged Nucleic Acids (BNA's), Zip Nucleic Acids (ZNA's), Minor Groove Binders (MGB's), Peptide Nucleic Acids (PNA's)) that provide for enhanced selectivity in hybridizing with particular alleles of interest. In general the modification increases the Tₘ difference between the exact matches and the single base mis-matches. Employing the combination of one or more modified primer(s) to selectively amplify one or more known alleles of a somatically mutated gene sequence and to selectively probe the amplification products to analyze the original sample for the presence of sparse populations of somatically mutated genes provides useful information for clinicians and the research community.

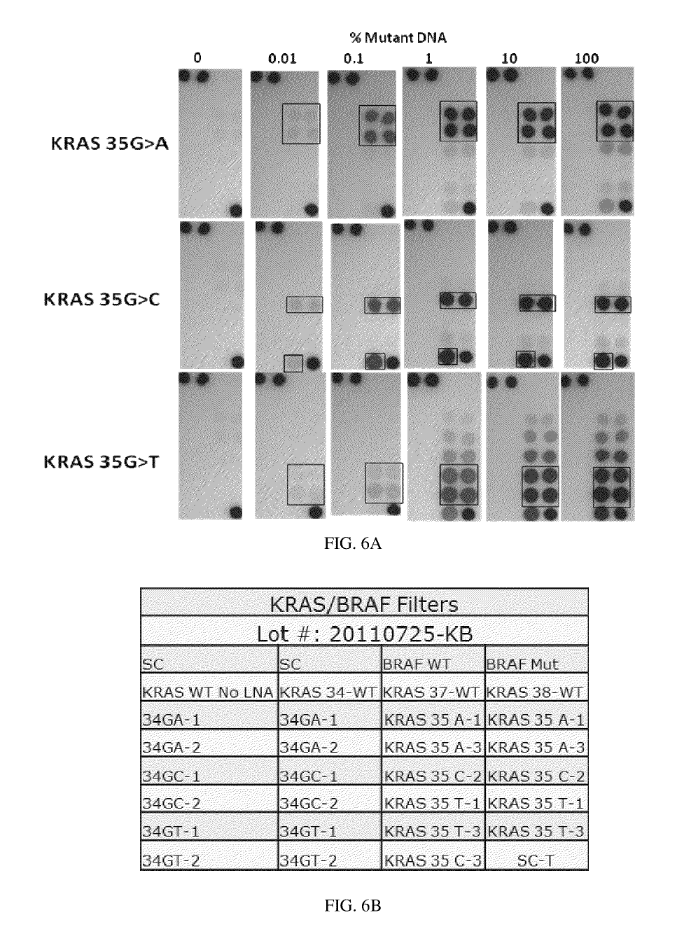

FIGS. 6A and 6B depict the microarray results and key for the experiment conditions described in FIGS. 4A-5B. For this example, the amount of “Mutant DNA” is a percentage of the template, ranging from 0% to 100% of mutant allele (either the KRAS G35A allele, KRAS G35C allele, or KRAS G35T allele), with the remaining percentage being the wild-type KRAS allele. The resulting amplicons are then run against a microarray with specific probes. For example, the mutant allele amplicons are to be detected with one or more mutant probes (e.g., KRAS 35 A-1, KRAS 35 A-3, KRAS 35 C-2, KRAS 35 T-1, KRAS 35 T-3), and the wild-type allele amplicons can be detected with a wild-type probe such as the “KRAS_WT_NoLNA,” “KRAS_{—}34 WT,” KRAS_{—}37 WT,” and “KRAS_{—}38 WT” probes. The results in FIG. 6A demonstrate that mutant amplicon is detected at as low as 0.01% of total DNA, which corresponds to 1 copy of mutant DNA in a background of 10,000 copies of wild-type DNA. See, for example, the boxed results in each of the three rows. Further, there is no detection of wild-type probes, meaning that the mutant primers selectively amplify the mutant allele without non-specifically amplifying the wild-type allele, despite the extremely high concentration of wild-type in many of the samples (including at 100% in column 1, and 99.99% in column 2). Using the LNA modified primers in conjunction with LNA modified probes, therefore, provides for a very strong analysis of only the desired mutated form of the allele even with nearly all of the DNA present in the original sample containing the wild-type non-mutated allele. As shown, the microarray provides a hybridization opportunity for any amplified wild-type allele to hybridize and since the modified primer sequence selected away from the wild-type allele even at wild-type allele concentration of 99.99% the only hybridization events present are for the selected mutated allele.

### Example 2

### KRAS G34X Analysis Using Modified Primers and Probes

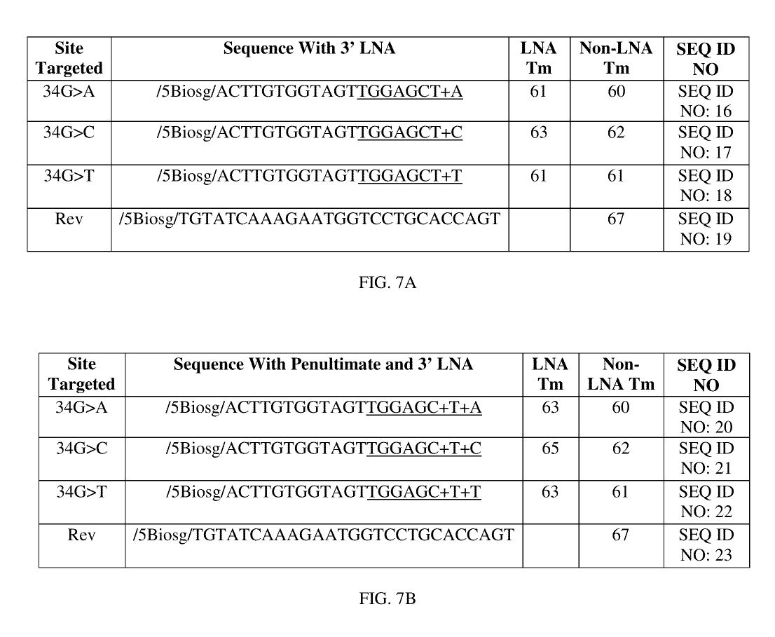

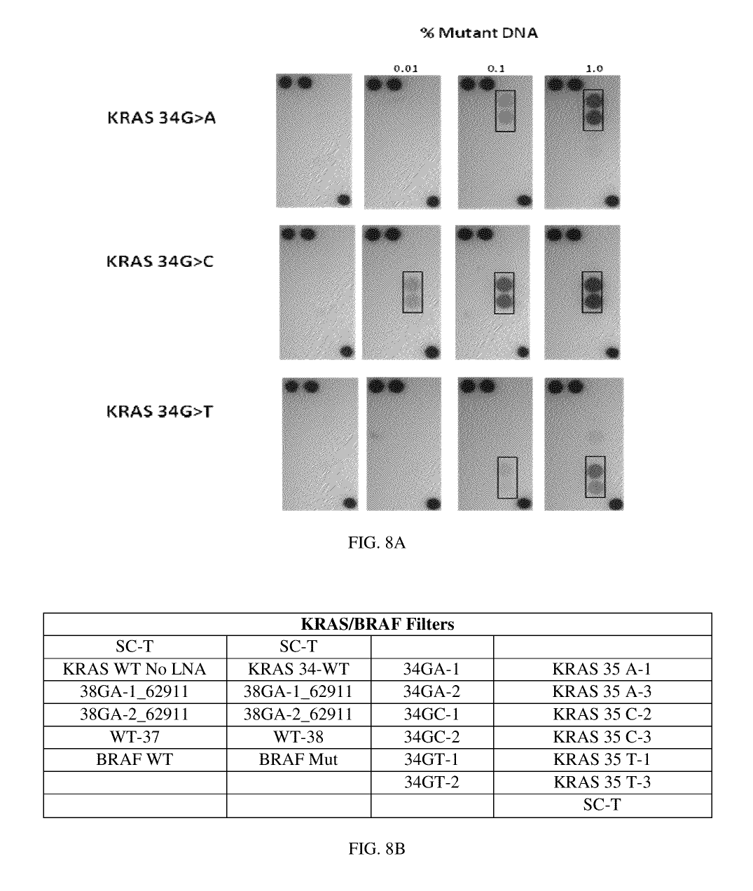

FIGS. 7A through 8B depict the conditions and results of a KRAS G34[A, C, T, or wild-type] assay using modified primers and modified probes according to an embodiment. This experiment confirms that the primer selection and modification system and method described above and in Example 1 can be applied to any somatically mutated allele against a background preponderance of non-mutated nucleic acid. In this example, the systems and methods are extended to the somatic mutation at site 34 of the KRAS gene where the 3′ terminal of the primer is modified with one or more LNAs.

In the assay described in Example 2, KRAS is amplified using a mixture of biotinylated KRAS_LNA PCR primers specific to the site of interest and biotinylated KRAS Rev primers. Capture probes with a wild-type “G” at the individual targeted positions are made using the coding strand sequence and will hybridize with the amplified biotinylated non-coding strand. Capture probes that will recognize mutant A, C, or T at the targeted positions are made using the non-coding strand sequence (therefore, T, G, or A at the respective sites) and will hybridize with the amplified biotinylated coding strand. According to one embodiment, the biotinylated LNA modified mutant primers and LNA modified capture probes are made to opposite strands. According to one embodiment, the biotinylated wild-type (generic primer) can be made off of the same strand as the mutant capture probes, and wild-type capture probe made off of same strand as the mutant primers.

As depicted in FIG. 7A, the following primers were used for amplification of a partial sequence within KRAS gene (the KRAS assay):

Forward Primers:
(SEQ ID NO: 16)
/5Biosg/ACTTGTGGTAGTTGGAGCT+A
for 34G > A detection;
(SEQ ID NO: 17)
/5Biosg/ACTTGTGGTAGTTGGAGCT+C
for 34G > C detection;
(SEQ ID NO: 18)
/5Biosg/ACTTGTGGTAGTTGGAGCT+T
for 34G > T detection;
Reverse Primer:
(SEQ ID NO: 19)
/5Biosg/TGTATCAAAGAATGGTCCTGCACCAGT
for all reactions.

where “/5Biosg/” represents a biotin, the “+[A, C, or T]” represents an LNA, and underlining indicates homology with the microarray probe. The Tm depicted in FIGS. 7A and 7B were calculated using traditional methods. According to a preferred embodiment, the modification of the 3′ terminal base with an LNA provides selective amplification of the mutation at site 34 while not amplifying the wild-type form of the allele.

In another embodiment, as depicted in FIG. 7B, the following primers were used for amplification of a partial sequence within KRAS gene (the KRAS assay):

Forward Primers:
(SEQ ID NO: 20)
/5Biosg/ACTTGTGGTAGTTGGAGC+T+A
for 34G > A detection;
(SEQ ID NO: 21)
/5Biosg/ACTTGTGGTAGTTGGAGC+T+C
for 34G > C detection;
(SEQ ID NO: 23)
/5Biosg/ACTTGTGGTAGTTGGAGC+T+T
for 34G > T detection;
Reverse Primer:
(SEQ ID NO: 23)
/5Biosg/TGTATCAAAGAATGGTCCTGCACCAGT
for all reactions.

where “/5Biosg/” represents a biotin, the “+[A, C, or T]” represents an LNA, and underlining indicates homology with the microarray probe. These primers/probes differ from those depicted in FIG. 7A in that there are two terminal LNAs rather than just one. As shown in the “LNA Tm” columns in FIGS. 7A and 7B, this additional LNA further affects the Tm of the primers.

As described above, the primers used for amplification of the KRAS allele in this example are modified by biotin and one or more LNAs, thereby increasing the melting temperature of an oligonucleotide (compare, for example, the “LNA Tm” and “Non-LNA Tm” columns in FIGS. 7A and 7B).

Although the primers used for amplification of the KRAS allele in this example are modified by biotin and one or more LNA, the primers and/or probes can be modified in a variety of other ways in order to increase hybridization. This includes, but is not limited to, the incorporation into the primer and/or probe of one or multiple LNAs, cLNA's, BNAs, ZNAs, MGBs, and/or PNAs, among many others. The beneficial result is that when a sample of cells that contain a somatic mutation are analyzed, only the allele(s) of interest is amplified and therefore subsequent analysis is facilitated since the material being analyzed only contains the allele(s) of interest.

FIGS. 8A and 8B depict the microarray results and key for the experiment conditions described in FIGS. 7A and 7B. For this example, the amount of “Mutant DNA” is a percentage of the template, ranging from 0% to 1% of mutant allele (either the KRAS G34A allele, KRAS G34C allele, or KRAS G34T allele), with the remaining percentage being the wild-type KRAS allele. The resulting amplicons are then run against a microarray with specific probes. For example, the mutant allele amplicons are to be detected with one or more mutant probes (e.g., 34GA-1, 34GA-2, 34GC-1, 34GC-2, 34GT-1, 34GT-2), and any wild-type allele amplicons can be detected with a wild-type probe such as the “KRAS WT No LNA,” “KRAS 34-WT,” “WT-37,” and “WT-38” probes. The results in FIG. 8A demonstrate that mutant amplicon is detected at as low as 0.01% of total DNA (see, for example, detection of KRAS G34C at 0.01%), which corresponds to 1 copy of mutant DNA in a background of 10,000 copies of wild-type DNA. Further, there is no detection of wild-type probes, meaning that the mutant primers selectively amplify the mutant allele without non-specifically amplifying the wild-type allele, despite the extremely high concentration of wild-type in many of the samples (including at 100% in column 1, and 99.99% in column 2). Using the LNA modified primers in conjunction with LNA modified probes, therefore, provides for a very strong analysis of only the desired mutated form of the allele even with nearly all of the DNA present in the original sample containing the wild-type non-mutated allele.

### Example 3

### BRAF T1799A and KRAS 38G>A Analysis Using Modified Primers and Probes

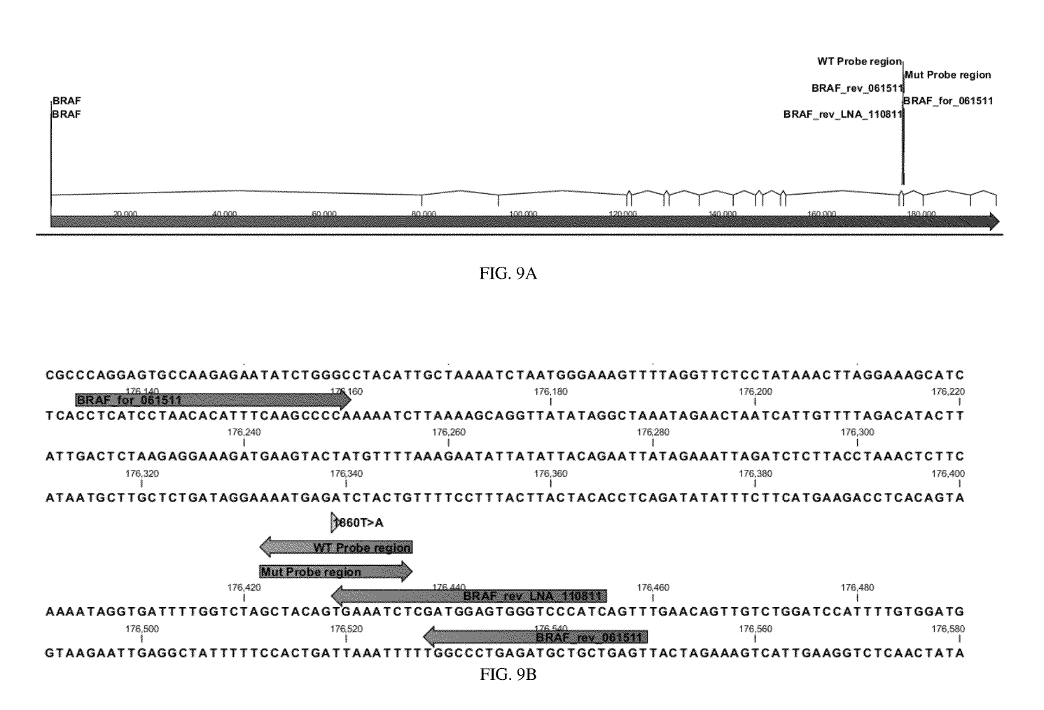

FIGS. 9 through 11 depict the conditions and results of a multiplex BRAF and KRAS 38G>A assay using modified primers and modified probes according to an embodiment. This example demonstrates yet again the universality of the systems and methods by analyzing the BRAF gene for the presence of particular know somatic mutations. The BRAF sequence shown in FIGS. 9A and 9B provides a targeted area where the somatic mutation occurs and the flanking sequences.

The BRAF gene, also known as “proto-oncogene B-Raf” and “v-Raf murine sarcoma viral oncogene homolog B1,” is a human gene that produces a protein called B-Raf. The B-Raf protein is involved in sending signals inside cells, which are involved in directing cell growth. BRAF is an oncogene, meaning that mutations in the BRAF gene can result or be otherwise involved in cancers such as non-Hodgkin lymphoma, colorectal cancer, malignant melanoma, papillary thyroid carcinoma, non-small-cell lung carcinoma, and/or adenocarcinoma of the lung, among others. To date, more than 30 different mutations of the BRAF gene associated with human cancers have been identified. The diagnosis of a mutation in the BRAF gene can be clinically important, since there are therapies available that target mutations in the gene.

In the assay described in this Example, BRAF is amplified using a mixture of reverse biotinylated BRAF_LNA primers specific to the site of interest, and biotinylated BRAF For primers, as shown in FIGS. 9A and 9B.

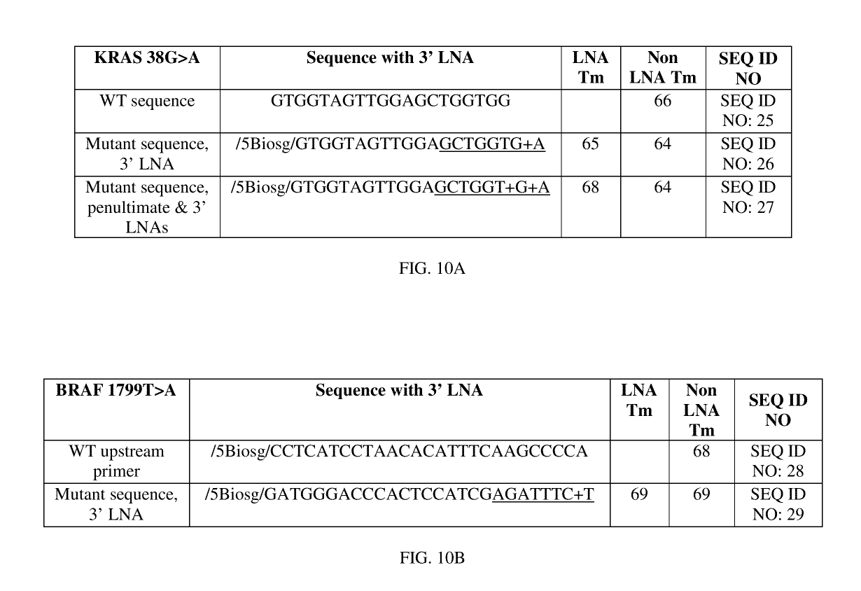

As depicted in FIG. 10B, the following primers were used for amplification of a partial sequence within the BRAF gene (the BRAF assay):

Forward Primer:
(SEQ ID NO: 28)
/5Biosg/CCT CAT CCT AAC ACA TTT CAA GCC CCA;
Reverse Primer:
(SEQ ID NO: 29)
/5Biosg/GATGGGACCCACTCCATCGAGATTTC+T;

where “5/Biosg/” represents a biotin, the “+[T]” represents an LNA and underlining indicates homology with the microarray probe. In this case the selective amplification primer is made using the non-coding strand sequence and the selective probe is made using the coding sequence.

As depicted in FIG. 10A, the following primers were used for amplification of a partial sequence within KRAS gene (the KRAS assay):

Forward Primers:
(SEQ ID NO: 25)
/5Biosg/GTGGTAGTTGGAGCTGGTG+A
for 38G > A detection;
(SEQ ID NO: 26)
/5Biosg/GTGGTAGTTGGAGCTGGT+G+A
for 38G > A detection;
Reverse Primer:
(SEQ ID NO: 27)
/5Biosg/TGTATCAAAGAATGGTCCTGCACCAGT;

where “/5Biosg/” represents a biotin, the “+[A, C, or T]” represents an LNA, and underlining indicates homology with the microarray probe. The Tm depicted in FIGS. 10A and 10B were calculated using traditional methods. According to a preferred embodiment, the modification of the 3′ terminal base with an LNA provides selective amplification of the mutation at site 38 while not amplifying the wild-type form of the allele.

As described above, the primers used for amplification of the KRAS and BRAF alleles in this example are modified by biotin and/or one or more LNAs, thereby increasing the melting temperature of an oligonucleotide (compare, for example, the “LNA Tm” and “Non-LNA Tm” columns in FIGS. 10A and 10B). Although the primers used for amplification of the KRAS and BRAF alleles in this example are modified by biotin and/or one or more LNA, the primers and/or probes can be modified in a variety of other ways in order to increase hybridization. This includes, but is not limited to, the incorporation into the primer and/or probe of one or multiple LNAs, cLNA's, BNAs, ZNAs, MGBs, and/or PNAs, among many others. The beneficial result is that when a sample of cells that contain a somatic mutation are analyzed, only the allele(s) of interest is amplified and therefore subsequent analysis is facilitated since the material being analyzed only contains the allele(s) of interest.

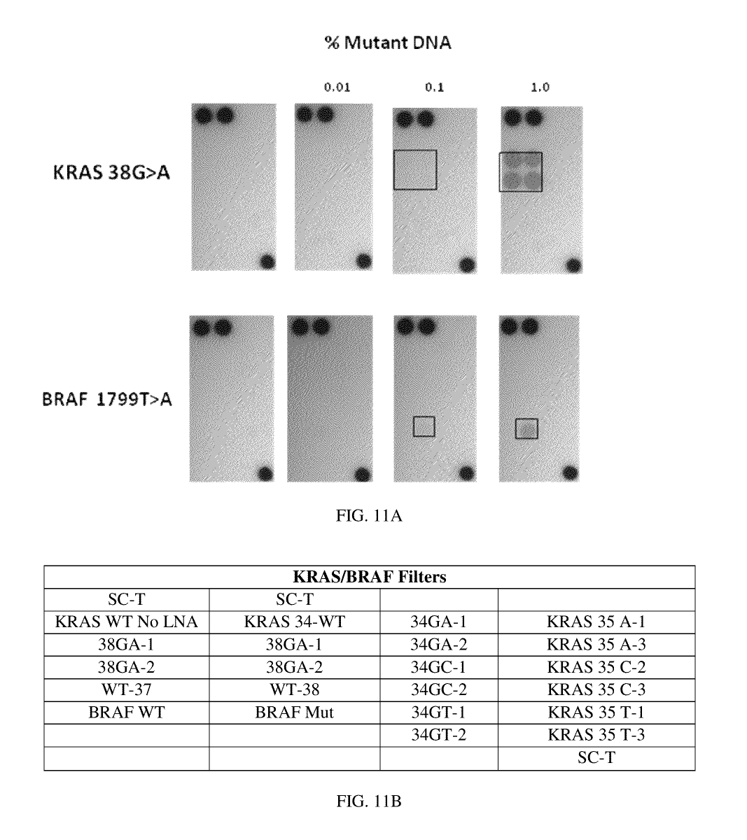

FIGS. 11A and 11B depict the microarray results and key for the experiment conditions described in FIGS. 9A through 10B. For this example, the amount of “Mutant DNA” is a percentage of the template, ranging from 0% to 1% of mutant allele (either the KRAS G38A allele or the BRAF 1799T>A allele), with the remaining percentage being the wild-type KRAS or BRAF allele. The resulting amplicons are then run against a microarray with specific probes. For example, the mutant allele amplicons are to be detected with one or more mutant probes (e.g., 38GA-1 and 38GA-2 for KRAS, and “BRAF Mut” for BRAF), and any wild-type allele amplicons can be detected with a wild-type probe such as the “KRAS WT No LNA,” “KRAS 34-WT,” “WT-37,” and “WT-38” probes for KRAS, and the “BRAF WT” probe for BRAF.

The results in FIG. 11A demonstrate that the mutant KRAS amplicon is detected at as low as 0.01% of total DNA (see, for example, detection of KRAS G38A at 0.1% in the top column), which corresponds to 1 copy of mutant DNA in a background of 1,000 copies of wild-type DNA. The mutant BRAF amplicon is detected at as low as 0.1% of total DNA (see, for example, detection of BRAF T1799A at 0.1% in the bottom column), which corresponds to 1 copy of mutant DNA in a background of 1,000 copies of wild-type DNA. Further, there is no detection by either of the wild-type probes, meaning that the mutant primers selectively amplify the mutant allele without non-specifically amplifying the wild-type allele, despite the extremely high concentration of wild-type in many of the samples (including at 100% in column 1, and 99.99% in column 2). Using the LNA modified primers in conjunction with LNA modified probes, therefore, provides for a very strong analysis of only the desired mutated form of the allele even with nearly all of the DNA present in the original sample containing the wild-type non-mutated allele.

Amplification and Analysis with Chemistry and Reagent Device

The methods and systems described herein can be conducted using a series of physically separated or different analytical or experimental devices, including: a first device or area for preparation of the sample (such as isolation and lysis of the cells) and/or purification of the nucleic acid; a second device for the PCR reaction; a third device comprising a microarray; a fourth device for detection of the microarray signal; and one or more computing devices for capturing, processing, analyzing, visualizing, or otherwise using data obtained from one or more of the analytical or experimental devices. In another embodiment where microarray analysis is replaced by another method of analysis, these experimental and/or detection devices will replace the microarray device listed above.

According to an aspect of the invention, the method is conducted in and/or on a single device capable of sample preparation, PCR, and detection of hybridization. In various embodiments, the device can include: a sample preparation component capable of receiving a biological sample and preparing the sample for PCR; a PCR component capable of receiving the sample from the sample preparation component and performing PCR on a nucleic acid target from the sample in order to produce the PCR results; a microarray component capable of receiving the target amplicon and detecting a hybridization event of the target amplicon to a probe bound to a surface of the microarray; and a support comprising the sample preparation component, the PCR component, and the microarray component.

According to an aspect of the invention, the method is conducted in an integrated microfluidic device known in the art, such as that disclosed in PCT Publication No. WO 2009/049268 A1 entitled “Integrated Microfluidic Device and Methods” by Peng Zhou et al., which is incorporated herein by reference. The method for detecting a nucleic acid target of interest in a sample disclosed herein can be readily adapted for use with an integrated microfluidic device, referred to as an assay unit or, commercially, as a CARD® (Chemistry and Reagent Device)), using methods known in the art, such as the methods disclosed in WO 2009/049268 A1.

“Microfluidics” generally refers to systems, devices, and methods for processing small volumes of fluids. Microfluidic systems can integrate a wide variety of operations for manipulating fluids. Such fluids may include chemical or biological samples. These systems also have many application areas, such as biological assays (for, e.g., medical diagnoses, drug discovery and drug delivery), biochemical sensors, or life science research in general as well as environmental analysis, industrial process monitoring and food safety testing. One type of microfluidic device is a microfluidic chip. Microfluidic chips may include micro-scale features (or “microfeatures”), such as channels, valves, pumps, reactors and/or reservoirs for storing fluids, for routing fluids to and from various locations on the chip, and/or for reacting reagents.

According to an aspect of the invention, the method is conducted in a self-contained, fully automated microfluidic device and system as disclosed in U.S. patent application Ser. No. 13/033,165 entitled “Self-Contained Biological Assay Apparatus, Methods, and Applications,” the entire contents of which are hereby incorporated herein by reference. The device comprises a self-contained, fully automated, biological assay-performing apparatus including a housing; a dispensing platform including a controllably-movable reagent dispensing system, disposed in the housing; a reagent supply component disposed in the housing; a pneumatic manifold removably disposed in the housing in a space shared by the dispensing platform, removably coupled to a fluidic transport layer and a plurality of reservoirs, wherein the fluidic transport layer, the reservoirs, and a test sample to be introduced therein are disposed in the housing in the space separate from the dispensing platform; a pneumatic supply system removably coupled to the pneumatic manifold in the housing in a space separate from the dispensing platform; and a control system coupled to at least one of the dispensing platform and the pneumatic supply system, disposed in the housing. The CARD dispensing platform can further include a motion control system operatively coupled to the reagent dispensing system, wherein the reagent dispensing system includes a reagent dispenser component having a distal dispensing end; and a camera connected to the reagent dispensing system having a field of view that includes at least a selected region of interest of the reservoirs.

According to another non-limiting aspect is an automated process for isolating, amplifying, and analyzing a target nucleic acid sequence using the CARD system. The process includes the steps of providing a pneumatic manifold that operates a microfluidic system having a fluidic transport layer and a fluidic channel disposed therein, and reservoirs attached thereto; introducing the fluid test sample into the fluidic channel; providing at least one reagent to the channel from at least one respective reservoir that is in fluid connection with the fluidic transport layer; combining the fluid test sample and the at least one reagent in a region of the fluidic transport layer, reservoir or amplification reactor; transporting the fluid test sample to a temperature-controlled amplification/reaction reactor that is in operative communication with the fluidic transport layer; incubating the fluid test sample in the amplification/reaction reactor under conditions sufficient to permit the target nucleic acid sequence to be amplified; transporting the fluid test sample to an analysis reservoir; and analyzing the amplified target nucleic acid sequence from the test sample, wherein the test sample is transported from a starting location in the fluidic transport layer to the analysis reservoir separately from any other samples and separately from the pneumatic manifold and the dispensing system.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening.

The recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. A method for selectively amplifying a target DNA sequence in the presence of non-target DNA sequence in a sample, the method comprising:
contacting an oligonucleotide system with the sample under hybridization conditions to form a reaction mixture, said oligonucleotide system including a forward primer and a reverse primer, wherein one of said forward or reverse primer is modified to preferentially increase hybridization between said primer and said target sequence, said modification comprising a modified 3′ terminal nucleotide;
cycling said oligonucleotide system so that, if the target DNA sequence is present in the sample, said forward primer and said reverse primer hybridize to the target DNA sequence and the reaction mixture results in a first amplified product; and
detecting the first amplified product, wherein said detecting step comprises use of a target DNA probe component for detecting said target DNA sequence, said target DNA probe component comprising a first modification, wherein said first modification preferentially increases hybridization between said target DNA probe component and said first amplified product.

2. The method of claim 1, wherein said modified 3′ terminal nucleotide is selected from the group consisting of locked nucleic acid, cLNA, bridged nucleic acid, zip nucleic acid, minor groove binder, peptide nucleic acid, and combinations thereof.

3. The method of claim 1, wherein said modified primer further comprises one or more additional modified nucleotides.

4. The method of claim 1, wherein the modified primer comprises the oligonucleotide sequence 5′-XYZ-3′, wherein:
X comprises one or more biotin groups;
Y comprises one or more nucleotides; and
Z comprises one or more modified nucleotides.

5. The method of claim 4, wherein Z comprises two consecutive locked nucleic acids at the 3′-terminus.

6. The method of claim 1, wherein the modified primer comprises the oligonucleotide sequence 5′-YZYZ-3′, wherein:
Y comprises one or more nucleotides; and
Z comprises one or more modified nucleotides.

7. The method of claim 1, wherein said target DNA sequence and at least some of said non-target DNA sequence in said sample differ by only one nucleic acid.

8. The method of claim 1, wherein the target DNA probe component comprises at least one modified nucleotide selected from the group consisting of locked nucleic acid, cLNA, bridged nucleic acid, zip nucleic acid, minor groove binder, peptide nucleic acid, and combinations thereof.

9. The method of claim 1, wherein the step of detecting the first amplified product comprises the steps of:
providing a microarray comprising a set of features including said target DNA probe component for detecting said target DNA sequence, and further including a component intended to serve as a positive control and a component intended to serve as a negative control;
contacting the microarray with said cycled reaction mixture to enable the first amplified product to bind to said target DNA probe component, wherein such binding results in the feature emitting the detectable signal; and
detecting said emitted detectable signal.

10. A method for selectively amplifying a target DNA sequence in the presence of non-target DNA sequence in a sample, the method comprising:
contacting an oligonucleotide system with the sample under hybridization conditions to form a reaction mixture, said oligonucleotide system including a forward primer and a reverse primer, wherein one of said forward or reverse primer is modified to preferentially increase hybridization between said primer and said target sequence, said modification comprising: (i) a modified 3′ terminal nucleotide, and (ii) one or more additional modified nucleotides;
cycling said oligonucleotide system so that, if the target DNA sequence is present in the sample, said forward primer and said reverse primer hybridize to the target DNA sequence and the reaction mixture results in a first amplified product;
providing a microarray comprising a set of features including at least a target DNA probe component for detecting said target DNA sequence, and further including a component intended to serve as a positive control and a component intended to serve as a negative control, wherein said target DNA probe component comprises a first modification, wherein said first modification preferentially increase hybridization between said target DNA probe component and said first amplified product;
contacting the microarray with said cycled reaction mixture to enable the first amplified product to bind to said target DNA probe component, wherein such binding results in the feature emitting the detectable signal; and
detecting said emitted detectable signal;
wherein said target DNA probe component comprises a first modification, wherein said first modification preferentially increases hybridization between said target DNA probe component and said first amplified product.

11. A system for selectively amplifying a target DNA sequence, the system comprising:
a sample comprising a non-target DNA sequence and potentially comprising said target DNA sequence;
an oligonucleotide system comprising a forward primer and a reverse primer under hybridization conditions to form a reaction mixture, wherein one of said forward or reverse primer is modified to preferentially increase hybridization between said primer and said target sequence, said modification comprising a modified 3′ terminal nucleotide;
a thermocycler adapted to cycle the oligonucleotide system so that, if the target DNA sequence is present in the sample, said forward primer and said reverse primer hybridize to the target DNA sequence and the reaction mixture results in a first amplified product;
a target DNA probe component for detecting said target DNA sequence, said target DNA probe component comprising a first modification, wherein said first modification preferentially increases hybridization between said target DNA probe component and said first amplified product; and
a detector adapted to detect the first amplified product.

12. The system of claim 11, wherein said modified 3′ terminal nucleotide is selected from the group consisting of locked nucleic acid, cLNA, bridged nucleic acid, zip nucleic acid, minor groove binder, peptide nucleic acid, and combinations thereof.

13. The system of claim 11, wherein said modified primer further comprises one or more additional modified nucleotides.

14. The system of claim 11, wherein the modified primer comprises the oligonucleotide sequence 5′-XYZ-3′, wherein:
X comprises one or more biotin groups;
Y comprises one or more nucleotides; and
Z comprises one or more modified nucleotides.

15. The system of claim 14, wherein Z comprises two consecutive locked nucleic acids at the 3′-terminus.

16. The system of claim 11, wherein said target DNA sequence and at least some of said non-target DNA sequence in said sample differ by only one nucleic acid.

17. The system of claim 11, wherein the target DNA probe component comprises at least one modified nucleotide selected from the group consisting of locked nucleic acid, cLNA, bridged nucleic acid, zip nucleic acid, minor groove binder, peptide nucleic acid, and combinations thereof.

18. The system of claim 11, wherein the detector is a microarray.

12. The system of claim 11, wherein the detector further comprises a component intended to serve as a positive control, and a component intended to serve as a negative control.

20. The system of claim 11, wherein the modified primer comprises the oligonucleotide sequence 5′-YZYZ-3′, wherein:
Y comprises one or more nucleotides; and
Z comprises one or more modified nucleotides.


SEQUENCE LISTING (37 sequences)

SEQ ID NO: 1 - DNA, 210 bp, Homo sapiens
  misc_feature (161)..(161): n is a, c, g, or t
agtgtcagcttcctctttcttgcctgggatctccctcctagtttcgtttctcttcctgttaggaattgttttcagcaatggaaagaaatggaaggagatccggcgtttctccctcatgacgctgcggaattttgggatggggaagaggagcattgaggacngtgttcaagaggaagcccgctgccttgtggaggagttgagaaaaaccaa

SEQ ID NO: 2 - DNA, 43 bp, Homo sapiens
  misc_feature (21)..(21): n is a, c, g, or t
ggaagaggagcattgaggacngtgttcaagaggaagcccgctg

SEQ ID NO: 3 - DNA, 347 bp, Homo sapiens
cgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgataca

SEQ ID NO: 4 - protein, 36 aa, Homo sapiens
TEYKLVVVGAGGVGKSALTIQLIQNHFVDEYDPTIE

SEQ ID NO: 5 - DNA, 187 bp, Homo sapiens
ttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgataca

SEQ ID NO: 6 - protein, 36 aa, Homo sapiens; identical to SEQ ID NO: 4

SEQ ID NO: 7 - DNA, 36 bp, Homo sapiens
aaacttgtggtagttggagctggtggcgtaggcaag

SEQ ID NO: 8 - protein, 12 aa, Homo sapiens
KLVVVGAGGVGK

SEQ ID NO: 9 - DNA, 20 bp, Artificial sequence
  Synthetic oligonucleotide primer, where N is a modified cysteine nucleotide
cttgtggtagttggagctgn

SEQ ID NO: 10 - DNA, 20 bp, Artificial sequence
  Synthetic oligonucleotide primer, where N is a modified thymine nucleotide
cttgtggtagttggagctgn

SEQ ID NO: 11 - DNA, 27 bp, Artificial sequence
  Synthetic oligonucleotide primer
tgtatcaaagaatggtcctgcaccagt

SEQ ID NO: 12 - DNA, 20 bp, Artificial sequence
  Synthetic oligonucleotide primer, where N1 is a modified guanine nucleotide and N2 is a modified adenine nucleotide
cttgtggtagttggagctnn

SEQ ID NO: 13 - DNA, 20 bp, Artificial sequence
  Synthetic oligonucleotide primer, where N1 is a modified guanine nucleotide and N2 is a modified cysteine nucleotide
cttgtggtagttggagctnn

SEQ ID NO: 14 - DNA, 20 bp, Artificial sequence
  Synthetic oligonucleotide primer, where N1 is a modified guanine nucleotide and N2 is a modified thymine nucleotide
cttgtggtagttggagctnn

SEQ ID NO: 15 - DNA, 27 bp, Artificial sequence
  Synthetic oligonucleotide primer
tgtatcaaagaatggtcctgcaccagt

SEQ ID NO: 16 - DNA, 20 bp, Artificial sequence
  Synthetic oligonucleotide primer, where N is a modified adenine nucleotide
acttgtggtagttggagctn

SEQ ID NO: 17 - DNA, 20 bp, Artificial sequence
  Synthetic oligonucleotide primer, where N is a modified cysteine nucleotide
acttgtggtagttggagctn

SEQ ID NO: 18 - DNA, 20 bp, Artificial sequence
  Synthetic oligonucleotide primer, where N is a modified thymine nucleotide
acttgtggtagttggagctn

SEQ ID NO: 19 - DNA, 27 bp, Artificial sequence
  Synthetic oligonucleotide primer
tgtatcaaagaatggtcctgcaccagt

SEQ ID NO: 20 - DNA, 20 bp, Artificial sequence
  Synthetic oligonucleotide primer, where NN is modified thymine and adenine nucleotides
acttgtggtagttggagcnn

SEQ ID NO: 21 - DNA, 20 bp, Artificial sequence
  Synthetic oligonucleotide primer, where NN is modified thymine and cysteine nucleotides
acttgtggtagttggagcnn

SEQ ID NO: 22 - DNA, 20 bp, Artificial sequence
  Synthetic oligonucleotide primer, where NN is modified thymine nucleotides
acttgtggtagttggagcnn

SEQ ID NO: 23 - DNA, 27 bp, Artificial sequence
  Synthetic oligonucleotide primer
tgtatcaaagaatggtcctgcaccagt

SEQ ID NO: 24 - DNA, 540 bp, Homo sapiens
cgcccaggagtgccaagagaatatctgggcctacattgctaaaatctaatgggaaagttttaggttctcctataaacttaggaaagcatctcacctcatcctaacacatttcaagccccaaaaatcttaaaagcaggttatataggctaaatagaactaatcattgttttagacatacttattgactctaagaggaaagatgaagtactatgttttaaagaatattatattacagaattatagaaattagatctcttacctaaactcttcataatgcttgctctgataggaaaatgagatctactgttttcctttacttactacacctcagatatatttcttcatgaagacctcacagtaaaaataggtgattttggtctagctacagtgaaatctcgatggagtgggtcccatcagtttgaacagttgtctggatccattttgtggatggtaagaattgaggctatttttccactgattaaatttttggccctgagatgctgctgagttactagaaagtcattgaaggtctcaactata

SEQ ID NO: 25 - DNA, 20 bp, Artificial sequence
  Synthetic oligonucleotide primer, where N is a modified adenine nucleotide
gtggtagttggagctggtgn

SEQ ID NO: 26 - DNA, 20 bp, Artificial sequence
  Synthetic oligonucleotide primer, where NN is modified guanine and adenine nucleotides
gtggtagttggagctggtnn

SEQ ID NO: 27 - DNA, 27 bp, Artificial sequence
  Synthetic oligonucleotide primer
tgtatcaaagaatggtcctgcaccagt

SEQ ID NO: 28 - DNA, 27 bp, Artificial sequence
  Synthetic oligonucleotide primer
cctcatcctaacacatttcaagcccca

SEQ ID NO: 29 - DNA, 27 bp, Artificial sequence
  Synthetic oligonucleotide primer, where N is a modified thymine nucleotide
gatgggacccactccatcgagatttcn

SEQ ID NO: 30 - DNA, 20 bp, Artificial sequence
  Synthetic oligonucleotide primer, where N1 is a modified thymine nucleotide and N2 is a modified adenine nucleotide
cttgtggtagttggagcngn

SEQ ID NO: 31 - DNA, 20 bp, Artificial sequence
  Synthetic oligonucleotide primer, where N1 is a modified cysteine nucleotide and N2 is a modified adenine nucleotide
cttgtggtagttggagntgn

SEQ ID NO: 32 - DNA, 20 bp, Artificial sequence
  Synthetic oligonucleotide primer, where N1 is a modified guanine nucleotide and N2 is a modified adenine nucleotide
cttgtggtagttgganctgn

SEQ ID NO: 33 - DNA, 20 bp, Artificial sequence
  Synthetic oligonucleotide primer, where N1 is a modified adenine nucleotide and N2 is a modified adenine nucleotide
cttgtggtagttggngctgn

SEQ ID NO: 34 - DNA, 20 bp, Artificial sequence
  Synthetic oligonucleotide primer, where N1 is a modified guanine nucleotide and N2 is a modified adenine nucleotide
cttgtggtagttgnagctgn

SEQ ID NO: 35 - DNA, 20 bp, Artificial sequence
  Synthetic oligonucleotide primer, where NNN is modified thymine, guanine, and adenine nucleotides
cttgtggtagttggagcnnn

SEQ ID NO: 36 - DNA, 20 bp, Artificial sequence
  Synthetic oligonucleotide primer, where NNNNNNN is modified guanine, adenine, guanine, cysteine, thymine, guanine, and adenine nucleotides
cttgtggtagttgnnnnnnn

SEQ ID NO: 37 - DNA, 20 bp, Artificial sequence
  Synthetic oligonucleotide primer, where N is a modified adenine nucleotide
cttgtggtagttggagctgn